(19) United States
(12) Patent Application Publication
(10) Pub. No.: US 2003/0065292 A1
(43) Pub. Date: Apr. 3, 2003

(54) Ozonated medical devices and methods of using ozone to prevent complications from indwelling medical devices

(76) Inventors: Rabih O. Darouiche, Houston, TX (US); David C. Shannon, Missouri City, TX (US)

Correspondence Address: BAKER BOTTS L.L.P., PATENT DEPARTMENT, 98 SAN JACINTO BLVD., SUITE 1500, AUSTIN, TX, 78701-4039, US

(21) Appl. No.: 10/164,184

(22) Filed: Jun. 5, 2002

Related U.S. Application Data
(60) Provisional application No. 60/296,837, filed on Jun. 8, 2001.

(51) Int. Cl. A61L 2/00; B32B 1/08; A61M 25/00

(52) U.S. Cl. 604/265; 427/2.1; 427/2.24; 428/36.91

(57) ABSTRACT

Indwelling medical devices resistant to microbial colonization and other complications include devices having a coating on one or more surfaces comprising an effective amount or concentration of an oxygen liberating substance, such as ozone, and optionally, one or more other therapeutic agents. Devices may alternately include a sleeve or other means which allows one or more surfaces of the device to be flushed or insufflated periodically with ozone or another oxygen liberating substance.

### CROSS REFERENCE TO RELATED APPLICATIONS

This application claims the benefit of U.S. Provisional Patent Application Ser. No. 60/296,837, filed Jun. 8, 2001 entitled “Ozonated Medical Devices and Methods of Using Ozone to Prevent Complications from Indwelling Medical Devices,” incorporated herein by reference in its entirety.

### BACKGROUND OF THE INVENTION

1. Field of the Invention

This invention relates to indwelling medical devices, and in particular, to the use of ozone and other oxygen liberating substances, alone, or in combination with other agents, for the prevention of infection and other complications from indwelling or invasive medical devices.

2. Description of the Background

Indwelling medical devices have been associated with a number of serious complications, including infection, malfunction, thrombosis, and inflammation, etc. Infection is the most common serious complication of indwelling medical devices. Microbial adherence to the surfaces of various medical devices can be very detrimental, as it can result in clinical infection and dysfunction of such devices. Although incorporation of traditional antimicrobial agents (antibiotics or antiseptics) onto the surfaces of medical devices has been used to provide some protection against bacterial colonization of the surfaces of indwelling medical devices, this approach is limited by the possibility that some organisms may have either inherent or induced resistance to these antimicrobial agents. Further, some antimicrobials are toxic, or otherwise not biocompatible.

Indwelling medical devices, such as intravenous and urinary catheters, can be essential in the management of hospitalized patients. However, the benefits derived from these catheters, as well as other types of invasive medical devices such as peritoneal catheters, cardiovascular devices, orthopedic implants and other prosthetic devices, may be offset by infectious complications. For example, the most common hospital-acquired infection is urinary tract infection (UTI). The majority of cases of UTI are associated with the use of urinary catheters, including transurethral foley, suprapubic and nephrostomy catheters. These urinary catheters are inserted in a variety of populations, including the elderly, stroke victims, spinal cord-injured patients, post-operative patients and those with obstructive uropathy. Despite adherence to sterile guidelines for the insertion and maintenance of urinary catheters, catheter-associated UTI continues to pose a major problem. For instance, it is estimated that almost one-quarter of hospitalized spinal cord-injured patients develop symptomatic UTI during their hospital course. Gram-negative bacilli account for almost 60-70%, enterococci for about 25% and Candida species for about 10% of cases of UTI.

Similarly, indwelling orthopedic devices are often associated with infection. About 5 to 20% of fracture fixation devices (pins, nails, screws, etc.) and about 1-3% of orthopedic joint implants become infected. Treatment of infected orthopedic implants, such as joint prostheses, usually requires both removal of the prosthesis and administration of a long course of antibiotics. In most cases, this is followed by re-implantation of a new joint prosthesis weeks or months later, after making sure that the infection has been eradicated.

A considerable amount of attention and study has been directed toward preventing microbial colonization of invasive medical devices by the use of antimicrobial agents, such as antibiotics, bound to the surface of the materials employed in such devices. In such attempts, the objective has been to produce a sufficient bacteriostatic or bactericidal action to prevent colonization.

Various methods have previously been employed to contact or coat the surfaces of medical devices with antimicrobials. For example, one method has been to flush the surfaces of the device with a solution of an antibiotic combination. Generally, contacting the surfaces by a simple flushing technique requires convenient access to the implantable device. For example, the interior surfaces of catheters are generally amenable to flushing with a solution of rifampin and minocycline or rifampin and novobiocin. For use in flushing solutions, the effective concentration of the antibiotic would range from about 1 to 10 μg/ml for minocycline, preferably about 2 μg/ml; 1 to 10 μg/ml for rifampin, preferably about 2 μg/ml; and 1 to 10 μg/ml for novobiocin, preferably about 2 μg/ml. The flushing solution would normally be composed of sterile water or sterile normal saline solutions.

Another known method of contacting or coating the surface of devices with antimicrobials would be to first apply or absorb to the surface of the medical device a layer of tridodecylmethyl ammonium chloride (TDMAC) surfactant followed by an antibiotic coating layer. For example, a medical device having a polymeric surface, such as polyethylene, silastic elastomers, polytetrafluoroethylene or Dacron®, can be soaked in a 5% by weight solution of TDMAC for 30 minutes at room temperature, air dried, and rinsed in water to remove excess TDMAC. Alternatively, TDMAC precoated catheters are commercially available. For example, central vascular catheters coated with TDMAC are available for patient use. The device carrying the absorbed TDMAC surfactant coating can then be incubated in an antibiotic solution for up to one hour or so, allowed to dry, then washed in sterile water to remove unbound antibiotic and stored in a sterile package until ready for implantation. In general, the antibiotic solution is composed of a concentration of 0.01 mg/ml to 60 mg/ml of each antibiotic in an aqueous pH 7.4-7.6 buffered solution, sterile water, or methanol. According to one method, an antibiotic solution of 60 mg of minocycline and 30 mg of rifampin per ml of solution is applied to the TDMAC-coated catheter.

A further method known to coat the surface of medical devices with antibiotics involves first coating the selected surfaces with benzalkonium chloride followed by ionic bonding of the antibiotic composition. See, e.g., Solomon, D. D. et al., J Controlled Release, 6:343-352 (1987) and U.S. Pat. No. 4,442,133.

Other methods of coating surfaces of medical devices with antibiotics are disclosed in U.S. Pat. No. 4,895,566 (a medical device substrate carrying a negatively charged group having a pKa of less than 6 and a cationic antibiotic bound to the negatively charged group); U.S. Pat. No. 4,917,686 (antibiotics are dissolved in a swelling agent which is absorbed into the matrix of the surface material of the medical device); U.S. Pat. No. 4,107,121 (constructing the medical device with ionogenic hydrogels, which thereafter absorb or ionically bind antibiotics); U.S. Pat. No. 5,013,306 (laminating an antibiotic to a polymeric surface layer of a medical device); and U.S. Pat. No. 4,952,419 (applying a film of silicone oil to the surface of an implant and then contacting the silicone film bearing surface with antibiotic powders).

Further, U.S. Pat. Nos. 5,624,704 and 5,902,283 disclose medical devices and methods for impregnating medical implants with antimicrobial agents so that the antimicrobial penetrates the material of the implants. U.S. Pat. Nos. 5,756,145 and 5,853,745 disclose durable antimicrobial coatings for implants, such as orthopedic implants, and methods of coating them. U.S. Pat. No. 5,688,516 describes compositions and methods of employing compositions to flush and coat medical devices, in which the compositions include combinations of a chelating agent, anticoagulant or antithrombotic agent with a non-glycopeptide antimicrobial agent.

Ozone has been shown to have a number of positive effects on a wide variety of physiologic processes. These include:

(1) Broad-spectrum antimicrobial activity against bacteria, protozoa, viruses, and fungi (Beuchat, et al., Lett Appl Microbiol, 29:202-5, 1999).

Ozone disrupts the integrity of the bacterial cell envelope through oxidation of the phospholipids and lipoproteins. In fungi, ozone inhibits cell growth at certain stages. With viruses, ozone damages the viral capsid and disrupts the reproductive cycle by disrupting the virus-to-cell contact with peroxidation. The weak enzyme coatings on cells which make them vulnerable to invasion by viruses make them susceptible to oxidation and elimination from the body, which then replaces them with healthy cells.

(2) Enhancement of circulation by inducing hypocoagulability of blood in patients with atherosclerosis (Maslennikov, et al., Klin Med, 75:35-7, 1997) and restoring flexibility/eliminating clumping of red blood cells.

In circulatory disease, clumping of red blood cells hinders blood flow through the small capillaries and decreases oxygen absorption due to reduced surface area. Ozone reduces or eliminates clumping and red cell flexibility is restored, along with oxygen carrying ability. Oxygenation of the tissues increases as the arterial partial pressure increases and viscosity decreases. Ozone also oxidizes the plaque in arteries, allowing the removal of the breakdown products and unclogging the blood vessels.

(3) Immunomodulating/anti-inflammatory properties for treatment of mandibular fractures (Malanchuk, et al., Klin Khir, 3:43-6, 2000), sciatic nerve pain (D'Erme, at al., Radiol Med [Torino], 95:21-4, 1998), and endophthalmitis (Gundarova, et al., Vestn Oftalmol, 112:9-11, 1996).

(4) Detoxifying property for use in hemotherapy (Ivanchenko S A., Lik Sprava, 7-8:130-3, 1999).

(5) Ozonotherapy for treatment of anaerobic soft tissue infections (Frantsuzov, et al., Khirurgiia, 10:21-3, 1999).

(6) Antioxidant property for reduction of injury caused by hepatic ischemia re-perfusion (Peralta, et al., Free Radic Res, 31:191-6, 1999).

A number of additional benefits have been attributed to ozone. For example, some have suggested that ozone stimulates the immune system, cleans arteries and veins, improves circulation, purifies the blood and lymph, normalizes hormone and enzyme production, reduces inflammation, reduces pain, calms the nerves, stops bleeding, prevents shock, prevents stroke damage, reduces cardiac arrhythmia, improves brain function and memory, oxidizes toxins allowing their excretion, chelates heavy metals, reverses degenerative diseases, prevents and treats communicable diseases, and prevents and eliminates auto-immune diseases.

Ozone is also believed to be useful in the stimulation of oxygen metabolism. Ozone causes an increase in the red blood cell glycolysis rate. This leads to the stimulation of 2,3-diphosphoglycerate (2,3-DPG) which leads to an increase in the amount of oxygen released to the tissues. There is a stimulation of the production of the enzymes which act as free radical scavengers and cell wall protectors: glutathione peroxidase, catalase, and superoxide dismutase. Ozone activates the Krebs cycle by enhancing oxidative carboxylation of pyruvate, stimulating production of ATP. Ozone also causes a significant reduction in NADH and helps to oxidize cytochrome C. Prostacyline, a vasodilator, is also induced by ozone.

In addition, ozone reacts with the unsaturated fatty acids of the lipid layer in cellular membranes, forming hydro peroxides. There is a synergistic effect with cellular-formed H₂O₂. Lipid peroxidation products include alkoxyl and peroxyl radicals, singlet oxygen, ozonides, carbonides, carbonyls, alkanes and alkenes.

Further, ozone may be useful in connection with the dissolution of malignant tumors. Ozone inhibits tumor metabolism. In addition, ozone oxidizes the outer lipid layer of malignant cells and destroys them through cell lysis (break-down). Phagocytes produce H₂O₂ and hydroxyl to kill bacteria and viruses. The generation of hydroxyl by killer cells is critical to their cytotoxic capability. Ozone stimulates conversion of L-arginine to citrulline, nitrite and nitrate by phagocytes, acting on tumors.

Ozone has previously been used as an antimicrobial, in connection with purification of water, and to inhibit microbial growth on certain other inanimate objects. Less frequently, ozone has been used in vivo, and has been administered to patients via several routes, including vascular, intramuscular, intradiscal, intraperitoneal, intraoral, intraocular, intraotic, and intrarectal (e.g., rectal insufflation) for various reasons.

Despite the many therapeutically beneficial effects of ozone, prior to the present invention, ozone has not been used in connection with indwelling, invasive devices to reduce or inhibit bacterial colonization and infection, nor to prevent or reduce other complications.

### SUMMARY OF THE INVENTION

The present invention overcomes the problems and disadvantages associated with current strategies and designs and provides an effective, economical and safe way to reduce infection and other complications from indwelling medical devices. The invention uses non-traditional agents that possess antimicrobial activity, such as ozone and other oxygen-liberating substances, to reduce or prevent complications due to indwelling or invasive medical devices.

The invention provides a practical, inexpensive, safe and effective method for coating, contacting or impregnating the material of various types of catheters and other medical implants with an oxygen liberating substance. It has surprisingly been discovered that by applying an ozone-containing agent to a catheter or other medical implant according to preferred embodiments of the invention, prolonged protection against a variety of bacterial and fungal organisms may be achieved. The invention is particularly useful for invasive devices which may be left in place in a patient for an extended period of time.

Accordingly, one embodiment of the invention is directed to a medical device resistant to microbial infection comprising an invasive device, and a coating on all or a portion of the invasive device. The coating comprises an effective amount of an oxygen liberating substance, which preferably is ozone. Preferably, the ozone (alone, or in combination with other agents) in gel or liquid form is used to coat the medical device before placing the device in the patient. For example, the ozone may be dissolved in olive oil (or any type of oil) to form a gel containing ozone bubbles, and the gel applied as a coating to the medical device.

In addition to ozone, the coating may further comprise an effective amount of another desirable agent which provides an additional therapeutic benefit, such as EDTA or trypsin. EDTA is commonly used as an anticoagulant, and also has inherent antimicrobial activity. Ozone also inhibits clotting. The combination of ozone and EDTA provides beneficial antimicrobial as well as anticoagulative properties. Trypsin breaks up biofilm, which develops on indwelling devices. Biofilm is subject to colonization by bacteria. The combination of ozone and trypsin potentiates the antimicrobial effect.

Another embodiment is directed to an invasive medical device system which resists infection comprising an invasive device, and an apparatus for insufflating or flushing at least one surface of the device with a fluid comprising an effective concentration of an oxygen liberating substance, such as ozone, while at least a portion of the invasive device is disposed in situ in a patient.

Another embodiment is directed to an invasive medical device comprising a medical device, at least a portion of which is designed or adapted to be placed in a patient's body; and an antimicrobial composition comprising an effective concentration of an oxygen liberating substance to inhibit the growth of microbial organisms. The antimicrobial composition coats the surface of, penetrates the exposed surface of, or impregnates the material forming at least a part of the portion of the medical device.

Another embodiment comprises a device for administering a therapeutic agent to a patient comprising: a catheter having a proximal end and a distal end, the distal end being adapted for insertion into a patient; a connector for fluidly connecting the proximal end of the catheter to a container containing the therapeutic agent; and an apparatus for providing an oxygen liberating substance to the connector.

Another embodiment is directed to a surgical implant comprising: an implantable device having an exterior; a cover around all or a portion of the exterior of the implantable device, the cover comprising a plurality of pores; and an apparatus for providing an oxygen liberating substance to the exterior of the implantable device, wherein a portion of the oxygen liberating substance passes through the pores and into the tissue or area surrounding the implant.

Still another embodiment of the invention is directed to a device for reducing infection at the point of entry of an invasive medical device into a patient comprising: a covering, the covering comprising a substrate and a source of an oxygen liberating substance, such as ozone.

Still another embodiment is directed to a method for reducing infection in an indwelling medical device comprising the steps of: providing an invasive medical device; and providing an effective amount of an oxygen liberating substance around all or a portion of the device. The step of providing an oxygen liberating substance may comprise applying a coating containing the oxygen liberating substance to at least a portion of the device, or, insufflating or flushing at least a portion of the area around the device with a gas or liquid comprising an oxygen liberating substance. In a preferred embodiment, a long-term indwelling venous or urinary catheter may be flushed by insufflating the catheter (in situ in the patient) with ozone gas periodically, e.g., one or more times a day, in order to reduce or inhibit bacterial growth.

Another embodiment is directed to a method for making an invasive medical device that is resistant to infection comprising the steps of forming an antimicrobial composition comprising an effective concentration of an oxygen liberating substance to inhibit the growth of microbial organisms relative to uncoated or untreated devices, and applying the oxygen containing composition to at least a portion of the medical device under conditions where the antimicrobial composition coats or permeates the material of the medical device.

Other embodiments and advantages of the invention are set forth in part in the description which follows, and in part, will be obvious from this description, or may be learned from the practice of the invention.

### DESCRIPTION OF THE DRAWINGS

FIG. 1 depicts an invasive medical device system according to one embodiment of the invention.

FIG. 2 is a perspective view of a device for administering a therapeutic agent according to another embodiment of the invention.

FIG. 3 is a cross sectional view of a surgical implant according to another embodiment of the invention.

### DETAILED DESCRIPTION OF THE INVENTION

As embodied and broadly described herein, the present invention relates to the use of ozone and other oxygen liberating substances to reduce infection and other complications from indwelling and other invasive medical devices. Specifically, the present invention is directed to the use of ozone (O₃) or another oxygen liberating substance (alone, or in combination with other therapeutic agents) to inhibit the growth of microorganisms on catheters and other indwelling medical devices.

As used herein, an “invasive device” or “invasive medical device” includes any device having a portion that may be placed percutaneously, transmucosally, surgically, or in any site beneath the skin or beneath or adjacent a mucous membrane. An “indwelling device” refers to an invasive device that is designed to be invasively placed in a patient and may be left in place for a period of time (e.g., more than one hour) sufficient to allow for microbial colonization.

As used herein, “oxygen liberating substances” refers to substances, such as ozone, that release or can be made to release oxygen at levels higher than in ambient air or water. Water normally includes only 7-20 ppm oxygen in diatomic (O₂) form. Air is typically between about 15-22% O₂. Oxygen liberating substances include, but are not limited to, ozone, medical ozone, mixtures of oxygen and ozone, hydrogen peroxide, chlorine dioxide and chlorite (ClO₂).

A preferred oxygen liberating substance for use in the invention is ozone. Ozone has been shown to have antimicrobial activities in non-medical applications. Ozone has also been used on inanimate objects in various applications (potential uses of ozone are described, for example, in U.S. Pat. Nos. 4,373,009; 6,174,326; 5,051,137; 4,746,489; 6,046,243; 4,778,456 and 6,190,407 B1; all of which are incorporated herein by reference). However, prior to the present invention, ozone has not been used to disinfect, sterilize or inhibit microbial growth in indwelling medical devices. In addition to its antimicrobial effects, ozone reduces clumping of blood cells, improves tissue oxygenation and reduces inflammation. As such, its use in medical devices provides a number of benefits to the patient.

It has been discovered that by applying an oxygen liberating substance, such as ozone, to the surface of a medical device, or impregnating the surface of a medical device with the oxygen liberating substance, the device can be made resistant to microbial infection.

In the practice of the invention, ozone (or another oxygen liberating substance) may be applied to the surface of a medical device in a variety of ways. For example, a coating may be applied on the surface of the device. The coating may be applied by any suitable means, such as by casting, spraying, painting, dipping, sponging, atomizing, smearing, impregnating, spreading, or other suitable means. One such embodiment of a medical device resistant to microbial infection comprises an invasive device, and a coating on all or a portion of the invasive device comprising an effective amount of an oxygen liberating substance. The oxygen liberating substance is preferably hydrogen peroxide, chlorine dioxide, chlorite, and more preferably, is ozone.

In a preferred embodiment, the medical device is coated with a gel containing ozone. A preferred gel for use in the invention may be made by ozonating olive oil. This may be accomplished, for example, by bubbling ozone through a carrier, such as olive oil, at approximately 4° C. for a period of time (e.g., several weeks). The oil foams and becomes a gel which is approximately 95% active as ozone gas. If refrigerated (e.g., prior to use), the gel will hold its ozone for a significant period of time. In addition to olive oil, ozone may alternately be bubbled in other carriers such as other oils, glycerol, or other organic agents, to form a gel. Ozone gel made from olive oil liquefies at room temperature. As such, liberation of oxygen from such gels according to the invention is enhanced at body temperatures.

Preferably, the gel or other coating formulation is composed so that the ozone is released over time. For example, the coating may be composed so that the ozone is released slowly over a period of approximately one day, more preferably, over a period of approximately three to four days, and most preferably, over a period of approximately three months. If desired, the coating may be composed so that the ozone or other oxygen liberating substance exerts its antimicrobial effect for periods exceeding three months.

Coatings according to the invention are not limited to gels, and may include, for example, liquids, emulsions, suspensions and solutions. If desired, ozone may be incorporated into collagen, gelatin, albumin, and other materials (e.g., biocompatible polymers) used to seal porous grafts and stents.

In addition to coatings, ozone may alternately be applied to the surface of a medical device by simply flushing the lumen of the device (e.g., a catheter) with a fluid containing an oxygen liberating substance, such as with ozone gas or ozone bubbled into the flushing solution. Alternately, ozone may be applied to the surface of a medical device by insufflating the area with ozone. For example, ozone may be instilled through a jacket (with holes or pores) that surrounds the internal and/or external surface of the indwelling device.

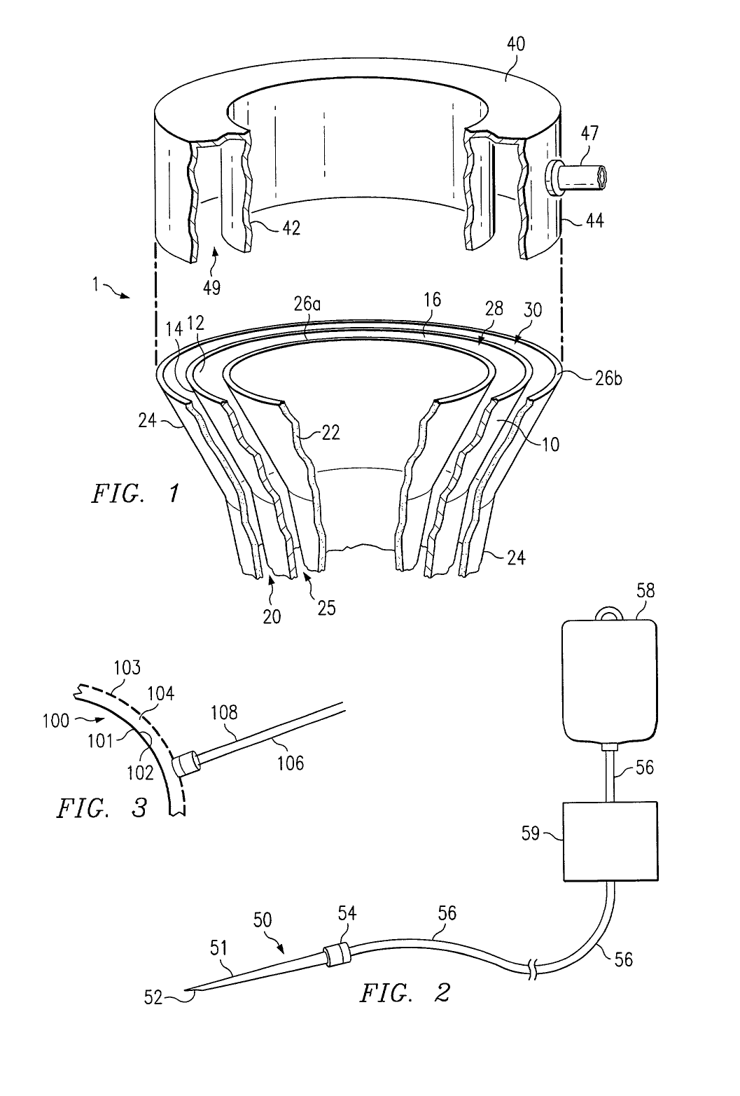

A preferred medical device system according to the invention in which ozone is applied to the surface of the device via a jacket or sleeve is depicted in FIG. 1. Specifically, as shown in FIG. 1, invasive medical device system 1 includes a catheter 10, which comprises an inner surface 12, an outer surface 14 and a hub 16. Catheter 10 is surrounded internally by porous inner sleeve 22, and externally by porous outer sleeve 24. Inner cylindrical space 25 is formed between porous inner sleeve 22 and inner surface 12 of catheter 10. Outer cylindrical space 20 is formed between porous outer sleeve 24 and outer surface 14 of catheter 10. Catheter 10 is disposed between spaces 25 and 20. Sleeves 22 and 24 each have hub region 26a and 26b, respectively, adjacent hub 16 of catheter 10.

As further shown in FIG. 1, porous outer sleeve 24 is disposed adjacent to and surrounds outer surface 14 of catheter 10. Porous inner sleeve 22 is disposed adjacent to and inside inner wall 12 of catheter 10. Preferably, sleeves 22 and 24 are made of a material such that they collapse onto the inner and outer walls of catheter 10 when not inflated, minimizing spaces 25 and 20. An opening 28, is formed between sleeve 22 and catheter 10 at hub 26a. An opening 30 is formed between sleeve 24 and catheter 10 at hub 26b.

Extension 40 is designed to mate with and provide a source of oxygen to spaces 25 and 20. Extension 40 comprises an inner wall 42 and an outer wall 44. Port 47 allows for the flow of ozone, or another desired oxygen liberating fluid (e.g., liquid or gas) into cylindrical space 49 formed between inner wall 42 and outer wall 44. Extension 40 is designed to mate with openings 28 and 30, such that ozone in space 49 flows through openings 28 and 30 into spaces 25 and 20, respectively.

In operation, extension 40 is attached at hubs 26a and 26b. Attachment may be accomplished by any suitable means or device known to those of skill in the art. Ozone is insufflated through port 47 into space 49. Ozone then flows through openings 28 and 30 into spaces 25 and 20, between sleeves 22 and 24 and catheter 10, thereby surrounding the inner wall and outer wall of catheter 10 with ozone. In addition, ozone also flows or bubbles through the pores (not shown) in sleeves 22 and 24 such that the interior surface of interior sleeve 22 and the outer surface of outer sleeve 24 are likewise exposed to ozone.

As can be seen from the foregoing, the design shown in FIG. 1 allows all surfaces of the catheter and sleeves to be flushed or insufflated with ozone or another oxygen liberating substance, thereby combating microbial infection.

The invention of FIG. 1 is not limited to catheters, but can be easily adapted to flush the surfaces (interior, exterior or both surfaces) of a number of medical devices, including, but not limited to, vascular catheters, urinary catheters, percutaneous devices, transmucosal devices, endotracheal tubes, surgically placed or implanted devices, and other suitable devices. If desired, a single sleeve (e.g., inner sleeve 22 or outer sleeve 24) may be used.

Accordingly, another embodiment of the invention is directed to an invasive medical device system which resists infection comprising an invasive device, and an apparatus for or means for insufflating or flushing at least one surface of the device with a fluid comprising an effective concentration of an oxygen liberating substance, while at least a portion of the invasive device is disposed in situ in a patient. The fluid may be a gas or liquid, and preferably, the oxygen liberating substance is ozone. Preferably, the at least one surface includes the exterior surface of the device (e.g., the outer wall of a catheter).

The apparatus for or means for insufflating or flushing preferably comprises a porous inner sleeve and a porous outer sleeve, wherein the at least a portion of the device is disposed between the porous inner sleeve and the porous outer sleeve. Alternately, the apparatus or means may comprise a single sleeve comprising a porous wall, wherein the at least a portion of the invasive device is disposed adjacent the porous wall. However, other devices and means may be used without departing from the spirit and scope of the invention.

Preferably, the invasive device is an indwelling vascular or urinary catheter.

In addition to coating, flushing or insufflating the invasive device, an oxygen liberating substance may be impregnated in the material actually used to make the medical device itself. This may be accomplished using the techniques described in U.S. Pat. Nos. 5,624,704 and 5,902,283 (incorporated herein by reference), or other suitable methods known to those of skill in the art.

As noted, ozone (or another oxygen liberating substance) may be used either alone, or it may be used in combination with other agents that may provide additional organ-specific benefits. For example, ozone may be combined with another therapeutic agent, such as trypsin, EDTA, steroids, NSAID's or antimicrobials. In such embodiments, the medical device provides additional therapeutic benefits such as reducing inflammation, improving oxygenation, reducing clotting, and reducing biofilm, among others.

In one such preferred embodiment, ozone is combined with one or more antimicrobial agents. As used herein, the term “antimicrobial agents” broadly includes, but is not limited to, antibiotics, antiseptics, disinfectants, antimicrobial peptides, synthetic moieties, and combinations thereof. Lipid and other complex formulations of antimicrobials as well as derivatives thereof can also be used. Antimicrobials that can be used in the practice of the invention include, but are not limited to, one or more of the antimicrobials disclosed in U.S. Pat. Nos. 5,624,704; 5,902,283; 5,756,145; 5,853,745; and 6,162,487 (all of which are incorporated by reference in their entirety).

Classes of antibiotics that may be used include, but are not limited to, tetracyclines (e.g. minocycline), rifamycins (e.g. rifampin), macrolides (e.g. erythromycin), penicillins (e.g. nafcillin), cephalosporins (e.g. cefazolin), other beta-lactam antibiotics (e.g. imipenem, aztreonam), aminoglycosides (e.g. gentamicin), chloramphenicol, sulfonamides (e.g. sulfamethoxazole), glycopeptides (e.g. vancomycin), quinolones (e.g. ciprofloxacin), fusidic acid, trimethoprim, metronidazole, clindamycin, mupirocin, polyenes (e.g. amphotericin B), azoles (e.g. fluconazole), beta-lactam inhibitors (e.g. sulbactam), streptogramins (e.g. quinupristin and dalfopristin), oxazolidinones (e.g. linezolid), lipopeptides (e.g. daptomycin), and ketolides. Examples of specific antibiotics that may be used include, but are not limited to, minocycline, rifampin, erythromycin, nafcillin, cefazolin, imipenem, aztreonam, gentamicin, sulfamethoxazole, vancomycin, ciprofloxacin, trimethoprim, metronidazole, clindamycin, teicoplanin, linezolid, daptomycin, dalbavancin, mupirocin, azithromycin, clarithromycin, ofloxacin, lomefloxacin, norfloxacin, nalidixic acid, sparfloxacin, pefloxacin, amifloxacin, enoxacin, fleroxacin, temafloxacin, tosufloxacin, gatifloxacin, moxifloxacin, gemifloxacin, clinafloxacin, sulbactam, clavulanic acid, amphotericin B, fluconazole, itraconazole, ketoconazole, and nystatin. Other examples of antibiotics, such as those listed in Sakamoto et al., U.S. Pat. No. 4,642,104, incorporated herein by reference, will readily suggest themselves to those of ordinary skill in the art.

Examples of useful antiseptics and disinfectants include, but are not limited to, thymol, α-terpineol, methylisothiazolone, cetylpyridinium, chloroxylenol, hexachlorophene, cationic biguanides (e.g. chlorhexidine, cyclohexidine), methylene chloride, iodine and iodophores (e.g. povidone-iodine), para-chloro-meta-xylenol, triclosan, furan medical preparations (e.g. nitrofurantoin, nitrofurazone), methenamine, aldehydes (glutaraldehyde, formaldehyde), taurinamides, alcohols, carboxylic acids and salts, and derivatives thereof. Other examples of antiseptics and disinfectants will readily suggest themselves to those of ordinary skill in the art.

The term “bacterial and fungal organisms” as used in the present invention includes all genuses and species of bacteria and fungi, including but not limited to, all spherical, rod-shaped and spiral bacteria. Preferably, medical devices according to preferred embodiments of the invention inhibit the growth of one or more microbial organisms when disposed in situ in a patient, selected from the group consisting of bacteria, fungi, protozoa or virus, for a period of approximately one day and, more preferably, for a period of approximately three to four days and, most preferably, for a period of approximately three months.

In another preferred embodiment, ozone may be combined with one or more antithrombotic/fibrinolytic agents, such as EDTA (ethylenediamine tetraacetic acid) and other calcium chelators, heparin chelators, or urokinase, etc. Useful antithrombotic/fibrinolytic agents in the practice of the invention include, but are not limited to, those described in U.S. Pat. No. 5,688,516 (incorporated herein by reference). The combination can be used to potentiate the anticoagulant and antimicrobial properties of ozone.

In another preferred embodiment, ozone is combined with one or more biofilm-disrupting agents. Biofilm develops on indwelling medical devices and facilitates colonization by bacteria. The combination of ozone with a biofilm-disrupting agent potentiates the anti-biofilm and antimicrobial properties of ozone. Biofilm disrupting agents include, for example, EDTA or another calcium chelator, or trypsin, etc. EDTA is an anticoagulant used in blood collection tubes. It is also recognized as a calcium chelating agent. EDTA is also recognized to have antibacterial effects (alone or in combination). Other chelating agents that may be used in conjunction with the present invention include, but are not limited to, EGTA (ethylene glycol-bis-[P-amino ethyl ether]-N, N, N′, N′-tetraacetic acid), DTPA (diethylenetriamine pentaacetic acid), DMSA, deferoxamine, Dimercaprol, edetate calcium disodium, TTH (triethylene tetramine dihydrochloride), zinc citrate, a combination of bismuth and citrate, penicillamine, succimer and Editronate. Other preferred chelating agents include, but are not limited to, those that chelate divalent metal cations such as Ca, Mg, Mn, Fe, Al, Pt, Ag, Au and Zn.

In another preferred embodiment, ozone is combined with one or more anti-inflammatory agents, including, for example, steroids or nonsteroidal anti-inflammatory drugs (NSAID's).

In another preferred embodiment, ozone is combined with two or more of any of the above listed agents, or is combined with another therapeutic agent.

Still another embodiment of the invention is directed to an invasive medical device comprising a medical device, at least a portion of which is designed to be invasively placed in a patient's body; and an antimicrobial composition comprising an effective concentration of an oxygen liberating substance to inhibit the growth of microbial organisms. The antimicrobial composition coats a surface of, penetrates an exposed surface of, or impregnates a material forming at least a part of the portion of the medical device. Preferably, the device inhibits the growth of microbial organisms for a period of at least one day, more preferably, at least three days, and most preferably, at least three months.

The amount of ozone used to coat, insufflate, flush or otherwise treat the devices of the invention will vary to some extent, but is at least a sufficient amount to form an effective concentration or amount to inhibit the growth of bacterial, fungal, protozoan and/or viral organisms. The terms “effective concentration” and “effective amount” as used in this application mean that a sufficient concentration or amount of the composition is added to achieve the desired therapeutic or other effect, e.g., to decrease, prevent or inhibit the growth of the target organisms. The actual amount or concentration used will vary based on factors such as the type of medical device, the age, sex, health and weight of the patient, and the use and length of use, as well as other factors known to those of skill in the art. As used herein, “patient” broadly includes, but is not limited to, a human or any animal being treated, tested or monitored in any kind of therapeutic, diagnostic, research, development or other application. Preferably, the patient is a human.

The use of oxygen liberating substances according to the invention may be applied to a wide variety of indwelling or invasive devices. Medical devices that can be used in the practice of the invention include vascular catheters, urinary catheters, other urinary devices, ventricular catheters, peritoneal dialysis and other peritoneal catheters, pleural catheters, catheters used to harvest bone marrow, wound drain tubes, vascular ports, hydrocephalus shunts, vascular and extravascular grafts, pacemaker systems and components, prosthetic heart valves, heart assist devices, penile prostheses and implants, breast implants, cosmetic implants, artificial sphincters, tissue bonding devices, bone prostheses, joint prostheses, small or temporary joint replacements, orthopedic implants, dental prostheses, dilators, stents, endotracheal tubes, tracheostomy devices, gastrotomy or intestinal tubes, biliary devices, maxillofacial implants, bioresorbable materials, ocular implants, ocular devices, otic devices, and soft tissue repair devices (including mesh), among others.

Vascular catheters include, but are not limited to, peripherally insertable central venous catheters, dialysis catheters, long-term tunneled central venous catheters, long-term untunneled central venous catheters, peripheral venous catheters, short-term central venous catheters, single-lumen and multiple-lumen central venous catheters, arterial catheters and pulnonary artery Swan-Ganz catheters.

The medical devices that can be used in the practice of the invention may be made of any desired material. These include, but are not limited to, non-metallic materials and metallic materials. Non-metallic materials may include thermoplastic or polymeric materials. Such materials include polyurethane, silicone, polyethylene, polyvinyl chloride, nylon, Gortex® (polytetrafluoroethylene), Dacron® (polyethylene tetraphthalate), Teflon®, latex, rubber, plastic, elastomers and materials that may be coated with gelatin, collagen, albumin, antimicrobial, antithrombotic/fibrinolytic agents, anti-inflammatory agents, biofilm-disrupting agents, hydrophilic agents, radioopaque agents, etc. Materials can be synthetic (listed above) or bioprosthetic, such as materials obtained from human (alloderm) or animal tissues (small intestinal submucosa or SIS), or combinations thereof.

Metallic materials include, but are not limited to, devices comprising stainless steel, titanium, titanium and other metal alloys. Particular metallic devices especially suited for application of the antimicrobial combinations of this invention include orthopedic implants such as joint prostheses, screws, nails, nuts, bolts, plates, rods, pins, wires, inserters, osteoports, halo systems and other orthopedic devices used for stabilization or fixation of spinal and long bone fractures or disarticulations. Other metallic devices may include non-orthopedic devices such as tracheostomy devices, dental prostheses, vascular devices, genitourinary implants, hepatobiliary implants, gastrointestinal devices, stylets, dilators, stents, wire guides and access ports of subcutaneously implanted vascular catheters.

The present invention is particularly useful with the use of tracheal devices (e.g., endotracheal tubes, tracheostomy tubes). The use of oxygen at high levels (e.g., over 60%) can be toxic. However, by substituting an ozone/oxygen mixture, toxicity can be reduced, while providing antimicrobial protection. The mixture may be provided continuously, if desired.

As will be clear to those of skill in the art, medical devices that can be used in the practice of the invention may be any of the medical devices described herein, or any other device adapted for invasive use, such as in a vessel, an organ, a digestive tract, a respiratory tract, a peritoneum, a pleural cavity, a thoracic cavity, a urinary tract, a hepatobiliary tract, a subcutaneous tissue, an intrathecal space, an ocular space, an otic space, and a bone or joint space, among others.

The present invention also includes devices useful for the in-line infusion of ozone or another oxygen liberating substance into a catheter or other indwelling medical device. For example, ozone may be infused in-line into a system which is being used to deliver an intravenous therapeutic agent via a vascular catheter into a patient. By bubbling or infusing ozone in-line into the intravenous fluid, it is possible to reduce or eliminate the unwanted transfer of bacterial and other contaminants into the patient's vascular system.

One such embodiment, shown in FIG. 2, comprises a device 50 for administering a therapeutic agent to a patient comprising: a catheter 51 having a proximal end (e.g. a hub) 54 and a distal end 52, the distal end being adapted for insertion into a patient; a connector 56 for fluidly connecting the proximal end of the catheter to a container 58 containing the therapeutic agent (e.g., a bag of fluids); and an apparatus 59 for providing ozone or another oxygen liberating substance to the connector. The connector may be any suitable device or means, and may comprise, for example, any kind of medical tubing suitable for use in intravenous infusion devices.

The catheter may be any of the various catheters described herein, but preferably is an intravenous or vascular catheter. The invention may be used to reduce microorganisms and other contaminants in any type of therapeutic agent or fluid, including, for example, intravenous fluids being used to provide total parenteral nutrition, whole blood and blood components being infused/transfused into a patient.

The ozone or other oxygen liberating substance may be infused or bubbled into the connector by any suitable apparatus. For example, the apparatus for providing ozone or another oxygen liberating substance may comprise a compartment or box which bubbles or infuses the ozone or oxygen liberating substance into the connector. Alternately, the apparatus for providing the ozone or oxygen liberating substance may comprise: a Y-tube in fluid communication with the connector; and a source of ozone or another oxygen liberating substance in fluid communication with the Y-tube. The oxygen liberating substance may be continuously, periodically, or intermittently bubbled or infused into the system.

Still another embodiment of the invention is directed to surgically implanted devices, such as orthopedic joint prosthesis, having a cover or coating which provides ozone or another oxygen liberating substance to the prosthetic device and the tissue surrounding the device. As noted, indwelling orthopedic devices are frequently associated with infection. In the event of infection, the device, such as a knee and hip prosthesis, typically must be removed and a replacement device implanted in the area where the infection occurred. Such replacement devices pose an increased risk of infection. However, the risk of infection may be reduced according to the invention by providing a source of ozone or another oxygen liberating substance to the replacement device and to the tissue surrounding the device.

Accordingly, one such embodiment of the invention is directed to a novel surgical implant (which may be the original or a replacement implant). As shown in FIG. 3, surgical implant 100 may comprise: an implantable device 101 having an exterior 102; a cover (coating or other suitable layer) 103 around all or a portion of the exterior of the implantable device, the cover comprising a plurality of pores 104; and an apparatus 106 for providing an oxygen liberating substance, such as ozone, to the exterior of the implantable device, wherein a portion of the oxygen liberating substance passes through the pores and into the tissue or area surrounding the implant. The apparatus for providing the oxygen liberating substance may be any suitable device or means, but in a preferred embodiment, it comprises a source of ozone (not shown) and a tube 108 in fluid communication with the exterior of the implantable device. In addition to providing infused or bubbled ozone to the implant and tissue around the implant, tube 108 also may serve the added function of a drain tube, e.g., it may provide drainage from the surgical site to the exterior of the patient for as long as the tube is in place. The tube may be a single poreless tube. Alternately, it may have pores, or it may include one or more insufflation sleeves, such as the device shown in FIG. 1. If desired, the cover may comprise a bioresorbable material. The cover may also be a removable sleeve. The implant device may be any type of surgical implant, and preferably, is an orthopedic prosthesis.

The invention may also be used to infuse ozone through cavities and pores in the implant itself.

Following placement of transmucosal and percutaneous catheters or other invasive devices, it is common to suture, tape or otherwise secure the proximal end or hub to the skin of the patient. A piece of gauze or other suitable adhesive covering (e.g., Tegaderm®) is typically placed over the top or hub of the catheter/device to minimize contamination from external sources. However, most organisms that infect percutaneous devices originate from the patient's own skin at the point of insertion of the device. By insufflating or flushing the gauze, Tegaderm® or other covering over the catheter/device with ozone or another oxygen liberating substance, the risk of contamination from organisms dwelling on the skin of the patient may be reduced. Additionally or alternately, a gel, ointment or other composition containing ozone or another oxygen liberating substance may be applied to the skin surrounding the point of insertion.

Accordingly, another embodiment of the invention is directed to a device for reducing infection at the point of entry of an invasive medical device into a patient comprising: a covering, the covering comprising a substrate and a source of an oxygen liberating substance, such as ozone. The device may also further include means for securing the covering to the skin of the patient. The substrate may be any suitable material, such as gauze, Tegaderm® or another material. The means for securing may include tape, an adhesive, or other suitable attachment mechanism. Preferably, the source of the oxygen liberating substance comprises an apparatus that insufflates, perfuses, flushes or infuses ozone into the covering and around skin at the point of insertion of the invasive device into the patient.

The use of ozone according to preferred embodiments of the invention prevents infection (the most common serious complication) of indwelling medical devices. In addition, the invention also may prevent other complications of indwelling medical devices, including malfunction, thrombosis, inflammation, etc. However, the use of ozone is not limited to medical devices, and can be used in a variety of applications. For example, oxygen liberating substances may be used to prevent colonization of the surfaces of industrial tubings (pipe lines, etc.) by a variety of pathogens, and to inhibit or prevent biofilm formation which may lead to obstruction of tubings. Ozone may also be used to prevent colonization of the surfaces of medical non-indwelling items (dental water lines, etc.) by a variety of pathogens and biofilm formation leading to obstruction of tubings. Further, the present invention is not limited to human medicine, but may be used in veterinary and any other application in which the antimicrobial and other benefits of the invention would be useful.

In addition to devices and systems, the present invention also includes methods of making and using the various devices of the invention. One such embodiment is directed to a method for reducing infection in an indwelling medical device comprising: providing an invasive medical device; and providing an effective amount of an oxygen liberating substance around all or a portion of the device. The step of providing an effective amount of an oxygen liberating substance may comprise applying a coating containing the oxygen liberating substance to at least a portion of the device. For example, the coating may comprise a gel containing ozone.

In a preferred embodiment, the coating is applied by casting, spraying, painting, dipping, sponging, atomizing, smearing, impregnating, spreading, or by other suitable methods. Preferably, the ozone is released over time.

Alternately, the step of providing an oxygen liberating substance may comprise flushing at least a portion of the surface of the device with a fluid (liquid or gas) comprising an oxygen liberating substance. Alternately, the step of providing an oxygen liberating substance may comprise insufflating at least a portion of the area around the device with a gas comprising an oxygen liberating substance. The area may be flushed or insufflated on an intermittent or periodic basis, e.g., every two to three hours, or once a day. Alternately, the oxygen liberating substance may be provided continuously.

In a preferred embodiment, chilled ozonated distilled water, saline solution, Ringer's solution, or other buffered solutions (e.g., chilled to 4° C.) is used as the flushing solution. The release of the ozone is enhanced at body temperatures (i.e., it comes out of solution), making this an ideal flushing solution for IV catheters.

Another embodiment of the invention is directed to a method for making an invasive medical device resistant to infection comprising the steps of: forming an antimicrobial composition comprising an effective concentration of an oxygen liberating substance to inhibit the growth of microbial organisms relative to, or as compared to, uncoated or untreated devices; and applying the oxygen containing composition to at least a portion of the medical device under conditions where the antimicrobial composition coats or permeates a material of the medical device. As with previous embodiments, the oxygen liberating substance is preferably ozone. The composition may further comprise trypsin, EDTA, a steroid, an NSAID, an antimicrobial or any of the other therapeutic agents described above. The step of applying may comprise casting, spraying, painting, dipping, sponging, atomizing, smearing, impregnating, spreading, or other suitable means.

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### Example 1

### Ozone Killing of Microorganisms in Solution

A clinical isolate of Escherichia coli strain 2131 that had caused catheter-related infection was used. The organism was grown overnight in tryptic soy broth (TSB) at 37° C., then diluted to a concentration of 10⁴ cfU/ml of normal saline. A 120 ml aliquot of the 10⁴ cfU/ml working bacterial suspension was placed in each of two beakers at 25° C.: (1) In the experimental arm, ozone was bubbled into the bacterial suspension; (2) In the control arm, no ozone was bubbled. Four hours later, three samples of the bacterial suspension in each beaker and serial dilutions were inoculated onto blood agar plates. Colony counts were determined at 24 hours after incubation of the agar plates at 37° C. The following table summarizes the results of cultures:

Number of E. coli colony forming units per ml (cfu/ml)

| | Experimental arm | Control arm |
|---|---|---|
| Sample #1 | 0 | 1000 |
| Sample #2 | 0 | 5000 |
| Sample #3 | 0 | 700 |

These results demonstrate that ozone kills E. coli in solution.

### Example 2

### Ozone Killing of Microorganisms on Catheter Surfaces

A clinical isolate of Escherichia coli strain 2131 that had caused catheter-related infection was used. The organism was grown overnight in tryptic soy broth (TSB) at 37° C., then diluted to a concentration of 10⁴ cfu/ml of TSB. The lumens of four 7-french, 20 -cm, triple-lumen polyurethane central venous catheters were filled with this working bacterial suspension and the catheters were then incubated at 25° C. for 4 hours. After draining the bacterial suspension from the lumens of catheters, the lumens were flushed with 1 ml of sterile normal saline. Using a closed dynamic flow system, the lumens of the four catheters were continually perfused for 4 hours at 25° C. with a recirculating total volume of 3 ml of sterile normal saline at a flow rate of 0.1 ml/minute. Ozone was continuously bubbled into the beaker containing the saline perfusing the lumens of two catheters (experimental arm), but no ozone was bubbled into the beaker containing the saline perfusing the lumens of the other two catheters (control arm). Four hours later, two 2 -cm segments from each of the four catheters (two in the experimental arm, and two in the control arm) were cultured onto blood agar plates using the sonication technique. Samples of the saline running through the experimental and control catheters were also inoculated onto blood agar plates. Colony counts were determined at 24 hours after incubation of the agar plates at 37° C. The following table summarizes the results of cultures of catheter segments:

Number of E. coli colony forming units cultured from
2-cm catheter segments

| | Experimental arm | Control arm |
|---|---|---|
| Segment #1 | 0 | 30 |
| Segment #2 | 0 | 30 |
| Segment #3 | 0 | 10 |
| Segment #4 | 0 | 0 |

These results demonstrate that ozone kills E. coli that has adhered to the catheter surface. Furthermore, ozone reduced the concentration of E. coli in the saline solution that was used to perfuse the infected lumens of catheters (9×10² cfu/ml vs. 42×10² cfu/ml).

Other embodiments and uses of the invention will be apparent to those skilled in the art from a consideration of the specification and practice of the invention disclosed herein. While the apparatus and methods of this invention have been described in terms of preferred embodiments, it will be apparent to those of skill in the art that variations may be applied to the apparatus and/or methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention. Not all embodiments of the invention will include all the specified advantages. The specification and examples should be considered exemplary only with the true scope and spirit of the invention indicated by the following claims.

What is claimed is:

1. A medical device resistant to microbial infection comprising:
an invasive device; and
a coating on all or a portion of the invasive device, the coating comprising an effective amount of an oxygen liberating substance.

2. The medical device of claim 1 wherein the oxygen liberating substance is selected from the group consisting of hydrogen peroxide, chlorine dioxide, chlorite and ozone.

3. The medical device of claim 1 wherein the oxygen liberating substance is ozone.

4. The medical device of claim 3 wherein the ozone is disposed in a gel.

5. The medical device of claim 4 wherein the gel comprises an organic agent.

6. The medical device of claim 4 wherein the gel comprises oil or glycerol.

7. The medical device of claim 1 wherein the coating further comprises an agent selected from the group consisting of trypsin, EDTA, a steroid, an NSAID, and an antimicrobial.

8. The medical device of claim 1 wherein the coating further comprises a chelating agent.

9. The medical device of claim 8 wherein the chelating agent is selected from the group consisting of EDTA, EGTA, DTPA, DMSA, deferoxamine, Dimercaprol, edetate calcium disodium, TTH, zinc citrate, a combination of bismuth and citrate, penicillamine, succimer and Editronate.

10. The medical device of claim 1 wherein the invasive device is selected from the group consisting of a vascular catheter, a urinary catheter, a urinary device, a ventricular catheter, a peritoneal dialysis catheter, a peritoneal catheter, a pleural catheter, a catheter used to harvest bone marrow, a wound drain tube, a vascular port, a hydrocephalus shunt, a vascular graft, an extravascular graft, a pacemaker system, a prosthetic heart valve, a heart assist device, a penile prosthesis, a breast implant, a cosmetic implant, an artificial sphincter, a tissue bonding device, a bone prosthesis, a joint prosthesis, a small joint replacement, a temporary joint replacement, an orthopedic implant, a dental prosthesis, a dilator, a stent, an endotracheal tube, a tracheostomy device, a gastrotomy tube, an intestinal tube, a biliary device, a maxillofacial implant, a bioresorbable material, an ocular implant, an ocular device, an otic device and a soft tissue repair device.

11. The medical device of claim 1 wherein the invasive device is a vascular catheter.

12. The medical device of claim 1 wherein the invasive device is adapted for use in a vessel, an organ, a digestive tract, a respiratory tract, a peritoneum, a pleural cavity, a thoracic cavity, a urinary tract, a hepatobiliary tract, a subcutaneous tissue, an intrathecal space, an ocular space, an otic space, a bone space or a joint space.

13. The medical device of claim 1 wherein the medical device inhibits the growth of one or more microbial organisms selected from the group consisting of bacteria and fungi.

14. The medical device of claim 1 wherein the medical device inhibits the growth of microbial organisms for a period of at least three months.

15. The medical device of claim 7 wherein the medical device provides a therapeutic benefit selected from the group consisting of reducing inflammation, improving oxygenation, reducing clotting and reducing biofilm.

16. An invasive medical device system which resists infection comprising:
an invasive device; and
an apparatus for insufflating or flushing at least one surface of the invasive device with a fluid comprising an effective concentration of an oxygen liberating substance while at least a portion of the invasive device is disposed in situ in a patient.

17. The system of claim 16 wherein the fluid is a gas.

18. The system of claim 16 wherein the oxygen liberating substance is ozone.

19. The system of claim 16 wherein the oxygen liberating substance is selected from the group consisting of hydrogen peroxide, chlorine dioxide, chlorite and ozone.

20. The system of claim 16 wherein the apparatus for insufflating or flushing comprises a sleeve comprising a porous wall, wherein the at least a portion of the invasive device is disposed adjacent the porous wall.

21. The system of claim 16 wherein the invasive medical device comprises a vascular catheter.

22. The system of claim 16 wherein the invasive medical device is selected from the group consisting of a vascular catheter, a urinary catheter, a transmucosal device, an endotracheal tube and a surgically placed device.

23. An invasive medical device comprising:
a medical device, at least a portion of which is designed to be placed in a patient's body; and
an antimicrobial composition comprising an effective concentration of an oxygen liberating substance to inhibit the growth of microbial organisms, wherein the antimicrobial composition coats a surface of, penetrates an exposed surface of, or impregnates a material forming at least a part of the portion of the medical device

24. The invasive medical device of claim 23 wherein the invasive medical device inhibits the growth of microbial organisms for a period of at least three months.

25. The invasive medical device of claim 23 wherein the oxygen liberating substance is ozone.

26. The invasive medical device of claim 23 wherein the oxygen liberating substance is selected from the group consisting of hydrogen peroxide, chlorine dioxide, chlorite and ozone.

27. A method for reducing infection in an indwelling medical device comprising:
providing an invasive medical device; and
providing an effective amount of an oxygen liberating substance around all or a portion of the device.

28. The method of claim 27 wherein the step of providing an effective amount of an oxygen liberating substance comprises applying a coating comprising the oxygen liberating substance to at least the portion of the device.

29. The method of claim 28 wherein the coating comprises a gel containing ozone.

30. The method of claim 28 wherein the coating is applied by casting, spraying, painting, dipping, sponging, atomizing, smearing, impregnating or spreading.

31. The method of claim 29 wherein the ozone is released over time.

32. The method of claim 27 wherein the step of providing an effective amount of an oxygen liberating substance comprises flushing at least a portion of the surface of the device with a fluid comprising an oxygen liberating substance.

33. The method of claim 32 wherein the fluid is a liquid or a gas.

34. The method of claim 27 wherein the oxygen liberating substance is ozone.

35. The method of claim 32 wherein the area is flushed on an intermittent or periodic basis.

36. The method of claim 32 wherein the area is flushed continuously.

37. The method of claim 27 wherein the step of providing an effective amount of an oxygen liberating substance comprises insufflating at least a portion of an area around the device with a gas comprising an oxygen liberating substance.

38. The method of claim 37 wherein the oxygen liberating substance is ozone.

39. The method of claim 37 wherein the area is insufflated on an intermittent or periodic basis.

40. A method for making an invasive medical device resistant to infection comprising the steps of:
forming an antimicrobial composition comprising an effective concentration of an oxygen liberating substance to inhibit the growth of microbial organisms relative to uncoated devices; and
applying the oxygen containing composition to at least a portion of the medical device under conditions where the antimicrobial composition coats or permeates a material of the medical device.

41. The method of claim 40 wherein the oxygen liberating substance is selected from the group consisting of hydrogen peroxide, chlorine dioxide, chlorite and ozone.

42. The method of claim 40 wherein the oxygen liberating substance is ozone.

43. The method of claim 40 wherein the composition further comprises an agent selected from the group consisting of trypsin, EDTA, a steroid, an NSAID and an antimicrobial.

44. The method of claim 40 wherein the step of applying comprises casting, spraying, painting, dipping, sponging, atomizing, smearing, impregnating, or spreading.

45. A device for administering a therapeutic agent to a patient comprising:
a catheter having a proximal end and a distal end, said distal end being adapted for insertion into the patient;
a connector for connecting the proximal end of the catheter to a container containing said therapeutic agent; and
an apparatus for providing an oxygen liberating substance to said connector.

46. The device of claim 45 wherein the oxygen liberating substance is ozone.

47. The device of claim 45 wherein the catheter is a vascular catheter.

48. The device of claim 45 wherein the therapeutic agent is whole blood, a blood component, or a fluid which provides parenteral nutrition.

49. The device of claim 45 wherein the apparatus for providing an oxygen liberating substance comprises a compartment which continuously, intermittently, or periodically bubbles or infuses ozone into the connector.

50. The device of claim 45 wherein the apparatus for providing an oxygen liberating substance comprises:
a Y-tube, the Y-tube being in fluid communication with said connector; and
a source of ozone in fluid communication with said Y-tube.

51. A surgical implant comprising:
an implantable device having an exterior;
a cover around all or a portion of said exterior of said implantable device, said cover comprising a plurality of pores; and
an apparatus for providing an oxygen liberating substance to said exterior of said implantable device, wherein a portion of said oxygen liberating substance passes through said pores and into a tissue surrounding the implant.

52. The implant of claim 51 wherein the oxygen liberating substance comprises ozone.

53. The implant of claim 51 wherein the implantable device comprises an orthopedic prosthesis.

54. The implant of claim 51 wherein said apparatus for providing an oxygen liberating substance comprises a tube in fluid communication with the exterior of said implantable device.

55. The implant of claim 51 wherein after implantation into a surgical site, said tube provides drainage from said surgical site.

56. The implant of claim 51 wherein the cover comprises a bioresorbable material or a removable sleeve.

57. A device for reducing infection at the point of entry of an invasive medical device into a patient comprising:
a covering, said covering comprising a substrate and a source of an oxygen liberating substance.

58. The device of claim 57 further comprising means for securing the covering to the skin of the patient.

59. The device of claim 57 wherein the source of the oxygen liberating substance comprises an apparatus that insufflates, perfuses, flushes or infuses ozone into the covering.
